(19) United States
(12) Patent Application Publication
(10) Pub. No.: US 2012/0238600 A1
(43) Pub. Date: Sep. 20, 2012

(54) TROPINONE BENZYLAMINES AS BETA-TRYPTASE INHIBITORS

(75) Inventors: Yong Mi CHOI-SLEDESKI, Belle Mead, NJ (US); Guyan LIANG, Warren, NJ (US); Patrick Wai-Kwok SHUM, Flemington, NJ (US)

(73) Assignee: SANOFI, Paris (FR)

(21) Appl. No.: 13/488,538

(22) Filed: Jun. 5, 2012

Related U.S. Application Data
(63) Continuation of application No. PCT/US2010/060004, filed on Dec. 13, 2010.
(60) Provisional application No. 61/289,574, filed on Dec. 23, 2009.

(30) Foreign Application Priority Data
Sep. 10, 2010 (FR) 1057199

(51) Int. Cl. A61K 31/46; A61P 11/00; C07D 451/02

(52) U.S. Cl. 514/304; 546/126; 546/125

(57) ABSTRACT

The present invention discloses and claims a series of substituted tropinone benzylamines of formula (I):

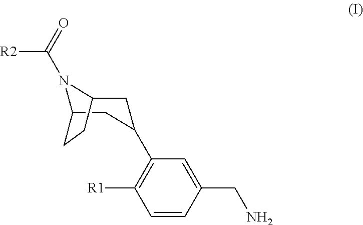

The compounds of this invention are inhibitors of β-tryptase and are, therefore, useful as pharmaceutical agents. Additionally, this invention also discloses methods of preparation of substituted tropinone benzylamines.

### BACKGROUND OF THE INVENTION

1. Field of the Invention

The present invention relates to a series of substituted tropinone benzylamines. The compounds of this invention are inhibitors of β-tryptase and are, therefore, useful as pharmaceutical agents. Additionally, this invention also relates to methods of preparation of substituted tropinone benzylamines and intermediates therefor.

2. Description of the Art

Mast cell mediated inflammatory conditions, in particular asthma, are a growing public health concern. Asthma is frequently characterized by progressive development of hyper-responsiveness of the trachea and bronchi to both immunospecific allergens and generalized chemical or physical stimuli, which lead to the onset of chronic inflammation. Leukocytes containing IgE receptors, notably mast cells and basophils, are present in the epithelium and underlying smooth muscle tissues of bronchi. These leukocytes initially become activated by the binding of specific inhaled antigens to the IgE receptors and then release a number of chemical mediators. For example, degranulation of mast cells leads to the release of proteoglycans, peroxidase, arylsulfatase B, chymase, and tryptase, which results in bronchiole constriction.

Tryptase is stored in the mast cell secretory granules and is the major protease of human mast cells. Tryptase has been implicated in a variety of biological processes, including degradation of vasodilatory and bronchodilatory neuropeptides (Caughey, et al., J. Pharmacol. Exp. Ther., 1988, 244, pages 133-137; Franconi, et al., J. Pharmacol. Exp. Ther., 1988, 248, pages 947-951; and Tam, et al., Am. J. Respir. Cell Mol. Biol., 1990, 3, pages 27-32) and modulation of bronchial responsiveness to histamine (Sekizawa, et al., J. Clin. Invest., 1989, 83, pages 175-179).

As a result, tryptase inhibitors may be useful as anti-inflammatory agents (K Rice, P. A. Sprengler, Current Opinion in Drug Discovery and Development, 1999, 2(5), pages 463-474) particularly in the treatment of chronic asthma (M. Q. Zhang, H. Timmerman, Mediators Inflamm., 1997, 112, pages 311-317), and may also be useful in treating or preventing allergic rhinitis (S. J. Wilson et al, Clin. Exp. Allergy, 1998, 28, pages 220-227), inflammatory bowel disease (S. C. Bischoff et al, Histopathology, 1996, 28, pages 1-13), psoriasis (A. Naukkarinen et al, Arch. Dermatol. Res., 1993, 285, pages 341-346), conjunctivitis (A. A. Irani et al, J. Allergy Clin. Immunol., 1990, 86, pages 34-40), atopic dermatitis (A. Jarvikallio et al, Br. J. Dermatol., 1997, 136, pages 871-877), rheumatoid arthritis (L. C. Tetlow et al, Ann. Rheum. Dis., 1998, 54, pages 549-555), osteoarthritis (M. G. Buckley et al, J. Pathol., 1998, 186, pages 67-74), gouty arthritis, rheumatoid spondylitis, and diseases of joint cartilage destruction.

In addition, tryptase has been shown to be a potent mitogen for fibroblasts, suggesting its involvement in the pulmonary fibrosis in asthma and interstitial lung diseases (Ruoss et al., J. Clin. Invest., 1991, 88, pages 493-499).

Therefore, tryptase inhibitors may be useful in treating or preventing fibrotic conditions (J. A. Cairns and A. F. Walls, J. Clin. Invest., 1997, 99, pages 1313-1321) for example, fibrosis, sceleroderma, pulmonary fibrosis, liver cirrhosis, myocardial fibrosis, neurofibromas and hypertrophic scars.

Additionally, tryptase inhibitors may be useful in treating or preventing myocardial infarction, stroke, angina and other consequences of atherosclerotic plaque rupture (M. Jeziorska et al, J. Pathol., 1997, 182, pages 115-122).

Tryptase has also been discovered to activate prostromelysin that in turn activates collagenase, thereby initiating the destruction of cartilage and periodontal connective tissue, respectively.

Therefore, tryptase inhibitors could be useful in the treatment or prevention of arthritis, periodontal disease, diabetic retinopathy, and tumour growth (W. J. Beil et al, Exp. Hematol., (1998) 26, pages 158-169). Also, tryptase inhibitors may be useful in the treatment of anaphylaxis (L. B. Schwarz et al, J. Clin. Invest., 1995, 96, pages 2702-2710), multiple sclerosis (M. Steinhoff et al, Nat. Med. (N.Y.), 2000, 6(2), pages 151-158), peptic ulcers and syncytial viral infections.

Such a compound should readily have utility in treating a patient suffering from conditions that can be ameliorated by the administration of an inhibitor of tryptase, e.g., mast cell mediated inflammatory conditions, inflammation, and diseases or disorders related to the degradation of vasodilatory and bronchodilatory neuropeptides, and have diminished liability for semicarbazide-sensitive amine oxidase (SSAO) metabolism.

β-tryptase is located solely in mast cell granules as the most abundant serine protease and is released following stimulation of the IgE receptor by allergen. In experimental animals, β-tryptase release provokes inflammation and bronchoconstriction characteristic of human asthma. It is also thought to cause fibroblast activation and therefore to have a role in airways remodeling. Levels of β-tryptase are elevated in bronchoalveolar lavage fluid (BALF) from asthmatic patients. Clinical proof-of-concept (bronchial allergen challenge) for asthma has been reported with an inhaled β-tryptase inhibitor (APC-366—since terminated due to bronchial irritation). β-tryptase inhibitors have the potential to impact the symptoms and pathogenesis of a number of proinflammatory indications, in particular, asthma and potentially COPD.

Benzylamine containing tryptase inhibitors, as one popular class of serine protease inhibitors, are also recognized as substrates for amine oxidases, especially SSAO.

All of the references described herein are incorporated herein by reference in their entirety.

Accordingly, it is an object of this invention to provide a series of substituted tropinone benzylamines that are inhibitors of β-tryptase.

It is also an object of this invention to provide processes for the preparation of the substituted tropinone benzylamines as disclosed herein.

Other objects and further scope of the applicability of the present invention will become apparent from the detailed description that follows.

### SUMMARY OF THE INVENTION

The present invention provides substituted tropinone benzylamines of formula I, and the stereoisomers, enantiomers, racemates and tautomers of said compounds and the pharmaceutically acceptable salts thereof, as inhibitors of β-tryptase, and methods of using the compounds of formula I as pharmaceutical agents for the treatment of diseases and disorders.

Thus in accordance with the practice of this invention there is provided a compound of formula (I):

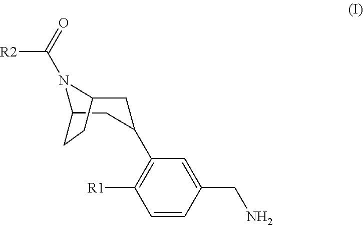

wherein
R1 is F, Cl, Br, OCH₂CO₂CH₃, CH₂OH, and other alkyl, haloalkyl and alkoxy, haloalkoxy groups; and
R2 is aryl or heteroaryl.

This invention further includes various salts of the compounds of formula (I) including various enantiomers or diastereomers of compounds of formula (I).

A further embodiment of the present invention relates to a method for inhibiting β-tryptase activity in a patient comprising administering to said patient a therapeutically effective amount of an inhibitor of β-tryptase.

Another embodiment of the present invention relates to a method for inhibiting β-tryptase activity in a patient comprising administering to said patient a therapeutically effective amount of a compound of formula I.

Another embodiment of the present invention relates to a method for treating a patient suffering from a disease or disorder ameliorated by inhibition of β-tryptase comprising administering to said patient a therapeutically effective amount of a compound of formula I.

In other aspects of this invention there are also provided various pharmaceutical compositions comprising one or more compounds of formula (I) as well as their therapeutic use in alleviating various diseases which are ameliorated by inhibition of β-tryptase.

### DETAILED DESCRIPTION OF THE INVENTION

The terms as used herein have the following meanings:

As used herein, the expression “(C₁-C₄)alkyl” includes methyl and ethyl groups, and straight-chained or branched propyl, and butyl groups. Particular alkyl groups are methyl, ethyl, n-propyl, isopropyl and tert-butyl. Derived expressions such as “(C₁-C₄)alkoxy”, “(C₁-C₄)alkoxy(C₁-C₄)alkyl”, or “hydroxy(C₁-C₄)alkyl” are to be construed accordingly.

As used herein, the expression “(C₁-C₆)perfluoroalkyl” means that all of the hydrogen atoms in said alkyl group are replaced with fluorine atoms. Illustrative examples include trifluoromethyl and pentafluoroethyl, and straight-chained or branched heptafluoropropyl, nonafluorobutyl, undecafluoropentyl and tridecafluorohexyl groups. Derived expression, “(C₁-C₆)perfluoroalkoxy”, is to be construed accordingly.

“Halogen” or “halo” means chloro, fluoro, bromo, and iodo.

As used herein, “patient” means a warm blooded animal, such as for example rat, mice, dogs, cats, guinea pigs, and primates such as humans.

As used herein, the expression “pharmaceutically acceptable carrier” means a non-toxic solvent, dispersant, excipient, adjuvant, or other material which is mixed with the compound of the present invention in order to permit the formation of a pharmaceutical composition, i.e., a dosage form capable of administration to the patient. One example of such a carrier can also be sterile water or pharmaceutically acceptable oil including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for parenteral administration.

The term “pharmaceutically acceptable salts” as used herein means that the salts of the compounds of the present invention can be used in medicinal preparations. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, hydrobromic acid, nitric acid, sulfamic acid, sulfuric acid, methanesulfonic acid, 2-hydroxyethanesulfonic acid, p-toluenesulfonic acid, fumaric acid, maleic acid, hydroxymaleic acid, malic acid, ascorbic acid, succinic acid, glutaric acid, acetic acid, propionic acid, salicylic acid, cinnamic acid, 2-phenoxybenzoic acid, hydroxybenzoic acid, phenylacetic acid, benzoic acid, oxalic acid, citric acid, tartaric acid, glycolic acid, lactic acid, pyruvic acid, malonic acid, carbonic acid or phosphoric acid. The acid metal salts such as sodium monohydrogen orthophosphate and potassium hydrogen sulfate can also be formed. Also, the salts so formed may present either as mono- or di-acid salts and can exist substantially anhydrous or can be hydrated. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts, and salts formed with suitable organic ligands, e.g. quaternary ammonium salts.

The expression “stereoisomers” is a general term used for all isomers of the individual molecules that differ only in the orientation of their atoms in space. Typically it includes mirror image isomers that are usually formed due to at least one asymmetric center, (enantiomers). Where the compounds according to the invention possess two or more asymmetric centers, they may additionally exist as diastereoisomers, also certain individual molecules may exist as geometric isomers (cis/trans). Similarly, certain compounds of this invention may exist in a mixture of two or more structurally distinct forms that are in rapid equilibrium, commonly known as tautomers. Representative examples of tautomers include keto-enol tautomers, phenol-keto tautomers, nitroso-oxime tautomers, imine-enamine tautomers, etc. It is to be understood that all such isomers and mixtures thereof in any proportion are encompassed within the scope of the present invention.

As used herein, ‘R’ and ‘S’ are used as commonly used terms in organic chemistry to denote specific configuration of a chiral center. The term ‘R’ (rectus) refers to that configuration of a chiral center with a clockwise relationship of group priorities (highest to second lowest) when viewed along the bond toward the lowest priority group. The term ‘S’ (sinister) refers to that configuration of a chiral center with a counterclockwise relationship of group priorities (highest to second lowest) when viewed along the bond toward the lowest priority group. The priority of groups is based upon sequence rules wherein prioritization is first based on atomic number (in order of decreasing atomic number). A listing and discussion of priorities is contained in Stereochemistry of Organic Compounds, Ernest L. Eliel, Samuel H. Wilen and Lewis N. Mander, editors, Wiley-Interscience, John Wiley & Sons, Inc., New York, 1994.

In addition to the (R)-(S) system, the older D-L system may also be used herein to denote absolute configuration, especially with reference to amino acids. In this system a Fischer projection formula is oriented so that the number 1 carbon of the main chain is at the top. The prefix ‘D’ is used to represent the absolute configuration of the isomer in which the functional (determining) group is on the right side of the carbon at the chiral center and ‘L’, that of the isomer in which it is on the left.

In a broad sense, the term “substituted” is contemplated to include all permissible substituents of organic compounds. In a few of the specific embodiments as disclosed herein, the term “substituted” means substituted with one or more substituents independently selected from the group consisting of (C₁-C₆)alkyl, (C₂-C₆)alkenyl, (C₁-C₆)perfluoroalkyl, phenyl, hydroxy, —CO₂H, an ester, an amide, (C₂-C₆)alkoxy, (C₁-C₆)thioalkyl, (C₁-C₆)perfluoroalkoxy, —NH₂, Cl, Br, I, F, —NH-lower alkyl, —N(lower alkyl)₂. However, any of the other suitable substituents known to one skilled in the art can also be used in these embodiments.

“Therapeutically effective amount” means an amount of the compound which is effective in treating the named disease, disorder or condition.

The term “treating” refers to:

(i) preventing a disease, disorder or condition from occurring in a patient that may be predisposed to the disease, disorder and/or condition, but has not yet been diagnosed as having it;

(ii) inhibiting the disease, disorder or condition, i.e., arresting its development; and

(iii) relieving the disease, disorder or condition, i.e., causing regression of the disease, disorder and/or condition.

Thus, in accordance with the practice of this invention there is provided a compound of the formula I:

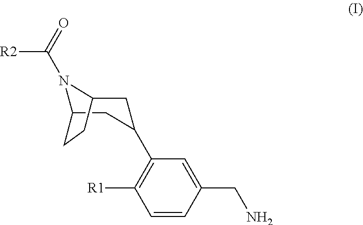

wherein
R1 is F, Cl, Br, OCH₂CO₂CH₃, CH₂OH, and other alkyl, haloalkyl and alkoxy, haloalkoxy groups; and
R2 is aryl or heteroaryl that is optionally substituted.

This invention further includes various salts of the compounds of formula (I) including various enantiomers or diastereomers of compounds of formula (I). As noted hereinabove and by way of specific examples hereafter all of the salts that can be formed including pharmaceutically acceptable salts are part of this invention. As also noted hereinabove and hereafter all of the conceivable enantiomeric and diastereomeric forms of compounds of formula (I) are part of this invention.

In one of the embodiments, there is provided the compounds of formula (I) wherein R1 is F, Cl, Br, OCH₂CO₂CH₃ or CH₂OH.

In another embodiment of this invention there is also provided a compound of formula (I), wherein

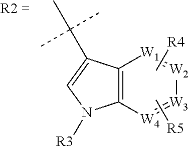

wherein
R3 is alkyl, or alkyl optionally substituted by one or more groups selected from hydroxy, alkoxy, haloalkoxy, cycloalkyl, heterocycle, aryl, optionally substituted aryl, heteroaryl or optionally substituted heteroaryl;
R4 and R5 are each independently H, halo, alkoxy, haloalkoxy, alkyl, amido, ureyl, carboxyl, sulfonyl amido, sulfonyl urea, alkyl optionally substituted by one or more groups selected from hydroxy, alkoxy, haloalkoxy, cycloalkyl, heterocycloalkyl, aryl and heteroaryl; and
W₁, W₂, W₃ or W₄ is N, CH, CR4 or CR5.

In yet another embodiment of this invention there is also provided a compound of formula (I), wherein R2 is indolyl or thiophenyl that is optionally substituted.

In a further aspect of this invention the following compounds encompassed by the scope of this invention without any limitation may be enumerated:
[3-(5-Aminomethyl-2-fluoro-phenyl)-8-aza-bicyclo[3.2.1]oct-8-yl]-[1-(2-methoxy-ethyl)-7-methyl-1H-indol-3-yl]-methanone hydrochloride;
[3-(5-Aminomethyl-2-fluoro-phenyl)-8-aza-bicyclo[3.2.1]oct-8-yl]-[1-(2-methoxy-ethyl)-7-trifluoromethoxy-1H-indol-3-yl]-methanone hydrochloride;
[3-(5-Aminomethyl-2-fluoro-phenyl)-8-aza-bicyclo[3.2.1]oct-8-yl]-[4-fluoro-1-(2-methoxy-ethyl)-7-methyl-1H-indol-3-yl]-methanone hydrochloride; and
[3-(5-Aminomethyl-2-fluoro-phenyl)-8-aza-bicyclo[3.2.1]oct-8-yl]-(4-bromo-3-methyl-5-propoxy-thiophen-2-yl)-methanone hydrochloride.

All of the above compounds may also include corresponding salts wherever possible including the pharmaceutically acceptable salts thereof.

This invention describes a novel alternative scaffold which can be used to generate a series of compounds with beta tryptase inhibitory activity. Based on the structure activity relationship (SAR) of piperidinyl benzylamines several P4 groups were chosen to determine whether this conformationally restricted scaffold would orient the P4 and P1 (benzylamine) groups such that the molecules would have utility as an inhibitor of a serine protease such as beta tryptase.

The compounds of this invention can be synthesized by any of the procedures known to one skilled in the art. Specifically, several of the starting materials used in the preparation of the compounds of this invention are known or are themselves commercially available. The compounds of this invention and several of the precursor compounds may also be prepared by methods used to prepare similar compounds as reported in the literature and as further described herein. For instance, see R. C. Larock, “Comprehensive Organic Transformations,” VCH publishers, 1989.

It is also well known that in various organic reactions it may be necessary to protect reactive functional groups, such as for example, amino groups, to avoid their unwanted participation in the reactions. Conventional protecting groups may be used in accordance with standard practice and known to one of skilled in the art, for example, see T. W. Greene and P. G. M. Wuts in “Protective Groups in Organic Chemistry” John Wiley and Sons, Inc., 1991. For example, suitable amine protecting groups include without any limitation sulfonyl (e.g., tosyl), acyl (e.g., benzyloxycarbonyl or t-butoxycarbonyl) and arylalkyl (e.g., benzyl), which may be removed subsequently by hydrolysis or hydrogenation as appropriate. Other suitable amine protecting groups include trifluoroacetyl [—C(═O)CF₃] which may be removed by base catalyzed hydrolysis, or a solid phase resin bound benzyl group, such as a Merrifield resin bound 2,6-dimethoxybenzyl group (Ellman linker) or a 2,6-dimethoxy-4-[2-(polystyrylmethoxy)ethoxy]benzyl, which may be removed by acid catalyzed hydrolysis, for example with TFA.

In another aspect of this embodiment, a specific disease, a disorder or a condition that can be prevented and/or treated with the compound of this invention include, without any limitation the following:

an inflammatory disease, for example, joint inflammation, including arthritis, rheumatoid arthritis and other arthritic condition such as rheumatoid spondylitis, gouty arthritis, traumatic arthritis, rubella arthritis, psoriatic arthritis, osteoarthritis or other chronic inflammatory joint disease, or diseases of joint cartilage destruction, ocular conjunctivitis, vernal conjunctivitis, inflammatory bowel disease, asthma, allergic rhinitis, interstitial lung diseases, fibrosis, scleroderma, pulmonary fibrosis, liver cirrhosis, myocardial fibrosis, neurofibromas, hypertrophic scars, various dermatological conditions, for example, atopic dermatitis and psoriasis, myocardial infarction, stroke, angina or other consequences of atherosclerotic plaque rupture, as well as periodontal disease, diabetic retinopathy, macular degeneration, acute macular degeneration, wet macular degeneration, tumor growth, anaphylaxis, multiple sclerosis, peptic ulcers, or a syncytial viral infection.

Tryptase is stored in the mast cell secretory granules and is the major secretory protease of human mast cells. Beta-tryptase has been implicated in a variety of biological processes, including degradation of vasodilating and bronchorelaxing neuropeptides (Caughey, et al., J. Pharmacol. Exp. Ther., 1988, 244, pages 133-137; Franconi, et al., J. Pharmacol. Exp. Ther., 1988, 248, pages 947-951; and Tam, et al., Am. J. Respir. Cell Mol. Biol., 1990, 3, pages 27-32) and modulation of bronchial responsiveness to histamine (Sekizawa, et al., J. Clin. Invest., 1989, 83, pages 175-179. As a result, tryptase inhibitors may be useful as anti-inflammatory agents (K Rice, P. A. Sprengler, Current Opinion in Drug Discovery and Development, 1999, 2(5), pages 463-474) particularly in the treatment of chronic asthma (M. Q. Zhang, H. Timrnerman, Mediators Inflarnm., 1997, 112, pages 311-317), and may also be useful in treating or preventing allergic rhinitis (S. J. Wilson et al, Clin. Exp. Allergy, 1998, 28, pages 220-227), inflammatory bowel disease (S. C. Bischoff et al, Histopathology, 1996, 28, pages 1-13), psoriasis (A. Naukkarinen et al, Arch. Dermatol. Res., 1993, 285, pages 341-346), conjunctivitis (A. A. Irani et al, J. Allergy Clin. Immunol., 1990, 86, pages 34-40), atopic dermatitis (A. Jarvikallio et al, Br. J. Dermatol., 1997, 136, pages 871-877), rheumatoid arthritis (L. C Tetlow et al, Ann. Rheum. Dis., 1998, 54, pages 549-555), osteoarthritis (M. G. Buckley et al, J. Pathol., 1998, 186, pages 67-74), gouty arthritis, rheumatoid spondylitis, and diseases of joint cartilage destruction. In addition, tryptase has been shown to be a potent mitogen for fibroblasts, suggesting its involvement in pulmonary fibrosis in asthma and interstitial lung diseases (Ruoss et al., J. Clin. Invest., 1991, 88, pages 493-499). Therefore, tryptase inhibitors may be useful in treating or preventing fibrotic conditions (J. A. Cairns and A. F. Walls, J. Clin. Invest., 1997, 99, pages 1313-1321) for example, fibrosis, sceleroderma, pulmonary fibrosis, liver cirrhosis, myocardial fibrosis, neurofibromas and hypertrophic scars.

Additionally, tryptase inhibitors may be useful in treating or preventing myocardial infarction, stroke, angina and other consequences of atherosclerotic plaque rupture (M. Jeziorska et al, J. Pathol., 1997, 182, pages 115-122).

Tryptase has also been discovered to activate prostromelysin that in turn activates collagenase, thereby initiating the destruction of cartilage and periodontal connective tissue, respectively.

Therefore, tryptase inhibitors could be useful in the treatment or prevention of arthritis, periodontal disease, diabetic retinopathy, and tumor growth (W. J. Beil et al, Exp. Hematol., (1998) 26, pages 158-169). Also, tryptase inhibitors may be useful in the treatment of anaphylaxis (L. B. Schwarz et al, J. Clin. Invest., 1995, 96, pages 2702-2710), multiple sclerosis (M. Steinhoff et al, Nat. Med. (N.Y.), 2000, 6(2), pages 151-158), peptic ulcers and syncytial viral infections.

Therefore, the compounds of this invention may have utility in the treatment of diseases or conditions ameliorated by inhibition of β-tryptase.

Thus in one aspect of this invention there is provided a method of treating a disease in a patient, said disease selected from the group consisting of: an inflammatory disease, for example, joint inflammation, including arthritis, rheumatoid arthritis and other arthritic condition such as rheumatoid spondylitis, gouty arthritis, traumatic arthritis, rubella arthritis, psoriatic arthritis, osteoarthritis or other chronic inflammatory joint disease, or diseases of joint cartilage destruction, ocular conjunctivitis, vernal conjunctivitis, inflammatory bowel disease, asthma, allergic rhinitis, interstitial lung diseases, fibrosis, scleroderma, pulmonary fibrosis, liver cirrhosis, myocardial fibrosis, neurofibromas, hypertrophic scars, various dermatological conditions, for example, atopic dermatitis and psoriasis, myocardial infarction, stroke, angina or other consequences of atherosclerotic plaque rupture, as well as periodontal disease, diabetic retinopathy, macular degeneration, acute macular degeneration, wet macular degeneration, tumor growth, anaphylaxis, multiple sclerosis, peptic ulcers, or a syncytial viral infection, comprising administering to said patient a therapeutically effective amount of a compound of formula (I).

One of skill in the art readily appreciates that the pathologies and disease states expressly stated herein are not intended to be limiting rather to illustrate the efficacy of the compounds of the present invention. Thus it is to be understood that the compounds of this invention may be used to treat any disease caused by the effects of β-tryptase. That is, as noted above, the compounds of the present invention are inhibitors of β-tryptase and may be effectively administered to ameliorate any disease state which is mediated all or in part by β-tryptase.

All of the various embodiments of the compounds of this invention as disclosed herein can be used in the method of treating various disease states as described herein. As stated herein, the compounds used in the method of this invention are capable of inhibiting the effects of β-tryptase and thereby alleviating the effects and/or conditions caused due to the activity of β-tryptase.

In another embodiment of the method of this invention, the compounds of this invention can be administered by any of the methods known in the art. Specifically, the compounds of this invention can be administered by oral, parenteral, intramuscular, subcutaneous, rectal, intratracheal, intranasal, intraperitoneal or topical route.

Finally, in yet another embodiment of this invention, there is also provided a pharmaceutical composition comprising a pharmaceutically acceptable carrier and a compound of formula (I), including enantiomers, stereoisomers, and tautomers of said compound and pharmaceutically acceptable salts, solvates or derivatives thereof, with said compound having the general structure shown in formula I as described herein.

As described herein, the pharmaceutical compositions of this invention feature β-tryptase inhibitory activity and thus are useful in treating any disease, condition or a disorder caused due to the effects of β-tryptase in a patient. Again, as described above, all of the preferred embodiments of the compounds of this invention as disclosed herein can be used in preparing the pharmaceutical compositions as described herein.

Preferably the pharmaceutical compositions of this invention are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. Alternatively, the compositions may be presented in a form suitable for once-weekly or once-monthly administration; for example, an insoluble salt of the active compound, such as the decanoate salt, may be adapted to provide a depot preparation for intramuscular injection. An erodible polymer containing the active ingredient may be envisaged. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Flavored unit dosage forms contain from 1 to 100 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The pharmaceutical compositions of this invention can be administered by any of the methods known in the art. In general, the pharmaceutical compositions of this invention can be administered by oral, intravenous, intramuscular, subcutaneous, rectal, intratracheal, intranasal, intraperitoneal or topical route. The preferred administrations of the pharmaceutical composition of this invention are by oral and intranasal routes. Any of the known methods to administer pharmaceutical compositions by an oral or an intranasal route can be used to administer the composition of this invention.

In the treatment of various disease states as described herein, a suitable dosage level is about 0.01 to 250 mg/kg per day, preferably about 0.05 to 100 mg/kg per day, and especially about 0.05 to 20 mg/kg per day. The compounds may be administered on a regimen of 1 to 4 times per day.

This invention is further illustrated by the following examples which are provided for illustration purposes and in no way limit the scope of the present invention.

### EXAMPLES

### General

As used in the examples and preparations that follow, the terms used therein shall have the meanings indicated: “kg” refers to kilograms, “g” refers to grams, “mg” refers to milligrams, “μg” refers to micrograms, “pg” refers to picograms, “lb” refers to pounds, “oz” refers to ounces, “mol” refers to moles, “mmol” refers to millimoles, “μmole” refers to micromoles, “nmole” refers to nanomoles, “L” refers to liters, “mL” or “ml” refers to milliliters, “μL” refers to microliters, “gal” refers to gallons, “° C.” refers to degrees Celsius, “R_{f}” refers to retention factor, “mp” or “m.p.” refers to melting point, “dec” refers to decomposition, “bp” or “b.p.” refers to boiling point, “mm of Hg” refers to pressure in millimeters of mercury, “cm” refers to centimeters, “nm” refers to nanometers, “abs.” refers to absolute, “conc.” refers to concentrated, “c” refers to concentration in g/mL, “DMSO” refers to dimethyl sulfoxide, “DMF” refers to N,N-dimethylformamide, “CU” refers to 1,1′-carbonyldiimidazole, “DCM” or “CH₂Cl₂” refers to dichloromethane, “DCE” refers to 1,2-dichloroethane, “HCl” refers to hydrochloric acid, “EtOAc” refers to ethyl acetate, “PBS” refers to Phosphate Buffered Saline, “IBMX” refers to 3-isobutyl-1-methylxanthine, “PEG” refers to polyethylene glycol, “MeOH” refers to methanol, “MeNH₂” refers to methyl amine, “N₂” refers to nitrogen gas, “iPrOH” refers to isopropyl alcohol, “Et₂O” refers to ethyl ether, “LAH” refers to lithium aluminum hydride, “heptane” refers to n-heptane, “HMBA-AM” resin refers to 4-hydroxymethylbenzoic acid amino methyl resin, “PdCl₂(dppf)₂” refers to 1,1′-bis(diphenylphosphino)ferrocene-palladium (II) dichloride DCM complex, “HBTU” refers to 2-(1H-benzotriazol-1yl)-1,1,3,3-tetramethyluronium hexafluorophosphate, “DIEA” refers to diisopropylethylamine, “CsF” refers to cesium fluoride, “MeI” refers to methyl iodide, “AcN,” “MeCN” or “CH₃CN” refers to acetonitrile, “TFA” refers to trifluoroacetic acid, “THF” refers to tetrahydrofuran, “NMP” refers to 1-methyl-2-pyrrolidinone, “H₂O” refers to water, “BOC” refers to t-butyloxycarbonyl, “brine” refers to a saturated aqueous sodium chloride solution, “M” refers to molar, “mM” refers to millimolar, “μM” refers to micromolar, “nM” refers to nanomolar, “N” refers to normal, “TLC” refers to thin layer chromatography, “HPLC” refers to high performance liquid chromatography, “HRMS” refers to high resolution mass spectrum, “L.O.D.” refers to loss on drying, “μCi” refers to microcuries, “i.p.” refers to intraperitoneally, “i.v.” refers to intravenously, anhyd=anhydrous; aq=aqueous; min=minute; hr=hour; d=day; sat.=saturated; s=singlet, d=doublet; t=triplet; q=quartet; m=multiplet; dd=doublet of doublets; br=broad; r.t.=room temperature; LC=liquid chromatograph; MS=mass spectrograph; ESI/MS=electrospray ionization/mass spectrograph; RT=retention time; M=molecular ion, “˜”=approximately.

Reactions generally are run under a nitrogen atmosphere. Solvents are dried over magnesium sulfate and are evaporated under vacuum on a rotary evaporator. TLC analyses are performed with EM Science silica gel 60 F254 plates with visualization by UV irradiation. Flash chromatography is performed using Alltech prepacked silica gel cartridges. The ¹H NMR spectra are run at 300 MHz on a Gemini 300 or Varian Mercury 300 spectrometer with an ASW 5 mm probe, and usually recorded at ambient temperature in a deuterated solvent, such as D₂O, DMSO-D₆ or CDCl₃ unless otherwise noted. Chemical shifts values (δ) are indicated in parts per million (ppm) with reference to tetramethylsilane (TMS) as the internal standard.

High Pressure Liquid Chromatography-Mass Spectrometry (LCMS) experiments to determine retention times (R_{T}) and associated mass ions are performed using one of the following methods:

Mass Spectra (MS) are recorded using a Micromass mass spectrometer. Generally, the method used was positive electro-spray ionization, scanning mass m/z from 100 to 1000. Liquid chromatography was performed on a Hewlett Packard 1100 Series Binary Pump & Degasser; Auxiliary detectors used were: Hewlett Packard 1100 Series UV detector, wavelength=220 nm and Sedere SEDEX 75 Evaporative Light Scattering (ELS) detector temperature=46° C., N₂ pressure=4 bar.
LCT: Grad (AcN+0.05% TFA):(H₂O+0.05% TFA)=5:95 (0 min) to 95:5 (2.5 min) to 95:5 (3 min). Column: YMC Jsphere 33×2 4 μM, 1 ml/min
MUX: Column: YMC Jsphere 33×2, 1 ml/min
Grad (AcN+0.05% TFA):(H2O+0.05% TFA)=5:95 (0 min) to 95:5 (3.4 min) to 95:5 (4.4 min).
LCT2: YMC Jsphere 33×2 4 μM, (AcN+0.05% TFA):(H2O+0.05% TFA)=5:95 (0 min) to 95:5 (3.4 min) to 95:5 (4.4 min)
QU: YMC Jsphere 33×2 1 ml/min, (AcN+0.08% formic acid):(H2O+0.1% formic acid)=5:95 (0 min) to 95:5 (2.5 min) to 95:5 (3.0 min)

The following examples describe the procedures used for the preparation of some of the compounds of this invention.

### Example 1

### [3-(5-Aminomethyl-2-fluoro-phenyl)-8-aza-bicyclo[3.2.1]oct-8-yl]-[1-(2-methoxy-ethyl)-7-methyl-1H-indol-3-yl]-methanone hydrochloride

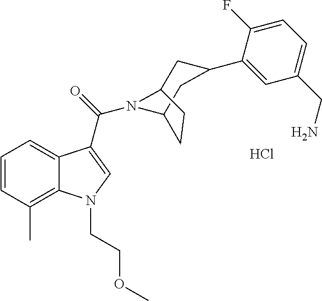

### Step A

### N-(3-bromo-4-fluoro-benzyl)-2,2,2-trifluoro-acetamide

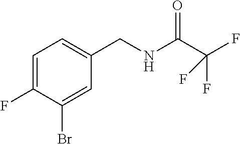

To a mixture of 3-bromo-4-fluoro-benzylamine hydrochloride (6.29 g, 26.2 mmol) in EtOAc (100 mL) at 0° C. was added TEA (4 mL, 28.8 mmol) dropwise over a 2 min period. After 10 min, TFAA (4.37 mL, 31.4 mmol) was added dropwise over a 2 min period. After this mixture was stirred at 0° C. for 2 h, it was partitioned between H₂O and EtOAc. The two layers were separated, and the organic layer was washed with sat NaHCO₃ and brine, dried over MgSO₄, filtered, and concentrated in vacuo. The crude material was purified on silica gel with heptane/EtOAc (50/50) as eluent to give 6.06 g (77%) of the product as a slightly yellow solid.

¹H NMR (CDCl₃, 300 MHz) δ 7.51 (dd, J=1.9, 6.3 Hz, 1H), 7.30-7.20 (m, 2H), 7.12 (t, J=12.5 Hz, 1H), 6.56 (bs, 1H), 4.49 (d, J=5.9 Hz, 2H);

¹⁹F-NMR (CDCl₃, 282 MHz) δ −75.32 (s, 3F), −107.00 (d, J=6.2 Hz, 1F);

LCMS 0.92 min m/z: [M+H]⁺=300.

### Step B

### 3-Trifluoromethanesulfonyloxy-8-aza-bicyclo[3.2.1]oct-2-ene-8-carboxylic acid tert-butyl ester

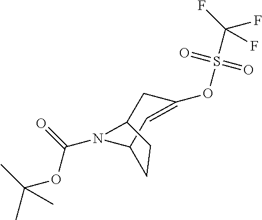

To a solution of potassium bis(trimethylsilyl)amide (60 mL, 30 mmol, 0.5 M in toluene) at −78° C. was added a solution of 3-oxo-8-aza-bicyclo[3.2.1]octane-8-carboxylic acid tert-butyl ester (6.1 g, 27.2 mmol) dropwise over a 10 min period. After 5 h, a solution of N-phenyl bistrifluoromethanesulfonamide (10.2 g, 28.7 mmol) in THF (10 mL) was added. After 5 h, the cooling bath was removed, and the mixture was stirred at rt for 2 h. The mixture was partitioned between H₂O and EtOAc. The two layers were separated, and the organic layer was washed with 1M NaOH and brine, dried over MgSO₄, filtered, and concentrated in vacuo to yield 7.6 g (78%) of the product as a clear colorless oil.

¹H NMR (CDCl₃, 300 MHz) δ 6.10 (d, J=4.2 Hz, 1H), 4.65-4.30 (m, 2H), 3.15-2.90 m, 1H), 2.35-1.90 (m, 4H), 1.85-1.50 (m, 2H), 1.46 (s, 9H);

¹⁹F NMR (CDCl₃, 282 MHz) δ −73.20 and −73.32 (total 3F).

### Step C

### 3-Trimethylstannanyl-8-aza-bicyclo[3.2.1]oct-2-ene-8-carboxylic acid tert-butyl ester

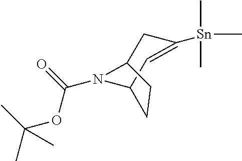

A mixture of 3-trifluoromethanesulfonyloxy-8-aza-bicyclo[3.2.1]oct-2-ene-8-carboxylic acid tert-butyl ester (4.17 g, 11.6 mmol), 1,1,1,2,2,2-hexamethyl-distannane (4.01 g, 12.2 mmol), anhydrous LiCl (0.52 g, 12.3 mmol), and tetrakistiphenylphosphinepalladium (0.67 g, 5% mol0 in degassed THF (30 mL) was heated at 80° C. for 6 h. The mixture was cooled to rt and then partitioned between H₂O and EtOAc. The two layers were separated, and the organic layer was washed with brine, dried over MgSO₄, filtered, and concentrated in vacuo. The crude material was purified on silica gel with heptane/EtOAc (100/0 to 70/30) as eluent to give 1.66 g (38%) of the product as a clear colorless oil.

¹H NMR (CDCl₃, 300 MHz) δ 6.10-5.95 (m, 1H), 4.30-4.05 (m, 2H), 2.95-2.65 (m, 1H), 2.20-1.95 (m, 1H), 1.90-1.70 (m, 2H), 1.65-1.50 (m, 1H), 1.37 (s, 9H), −0.07 (s, 9H).

### Step D

### 3-{2-Fluoro-5-[(2,2,2-trifluoro-acetylamino)-methyl]-phenyl}-8-aza-bicyclo[3.2.1]oct-2-ene-8-carboxylic acid tert-butyl ester

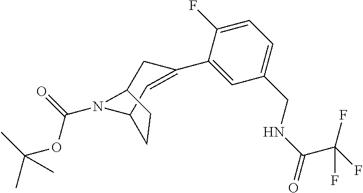

A mixture of 3-trimethylstannanyl-8-aza-bicyclo[3.2.1]oct-2-ene-8-carboxylic acid tert-butyl ester (1.66 g, 4.46 mmol), N-(3-bromo-4-fluoro-benzyl)-2,2,2-trifluoro-acetamide (1.61, 5.35 mmol), and tetrakis(triphenylphosphine)palladium (0) (0.26 g, 5% mol in degassed toluene (50 mL) was heated at 110° C. overnight. The mixture was cooled to rt and then partitioned between H₂O and EtOAc. The two layers were separated, and the organic layer was washed with brine, dried over MgSO₄, filtered, and concentrated in vacuo. The crude material was purified on silica gel with heptane/EtOAc (80/20 to 50/50) as eluent to give 1.33 g (69%) of the product as a clear colorless sticky gum.

¹H NMR (CDCl₃, 300 MHz) δ 7.20-7.10 (m, 2H), 7.05-6.95 (m, 1H), 6.63 (bs, 1H), 4.60-4.30 (m, 4H), 3.20-3.00 (m, 1H), 2.35-2.10 (m, 2H), 2.10-1.90 (m, 2H), 1.90-1.70 (m, 1H), 1.60-1.50 (m, 1H), 1.47 (s, 9H);

¹⁹F NMR (CDCl₃, 282 MHz) δ −75.32 (s, 3F), −114.02 (s, 1F);

LCMS 8.39 min m/z: [M+H]⁺=429.

### Step E

### 3-{2-Fluoro-5-[(2,2,2-trifluoro-acetylamino)-methyl]-phenyl}-8-aza-bicyclo[3.2.1]octane-8-carboxylic acid tert-butyl ester

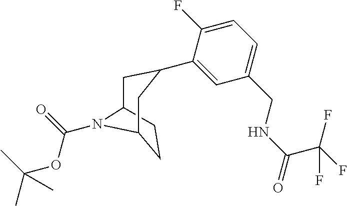

A mixture of 3-{2-fluoro-5-[(2,2,2-trifluoro-acetylamino)-methyl]-phenyl}-8-aza-bicyclo[3.2.1]oct-2-ene-8-carboxylic acid tert-butyl ester (4.87 g, 11.4 mmol) and 10% Pd/C (1.0 g) in MeOH (100 mL) was hydrogenated at 60 psi for 9 h. The mixture was filtered through Celite, and the filtrate was concentrated in vacuo. The residue was redissolved in CH₂Cl₂, dried over MgSO₄, filtered, and concentrated in vacuo to yield 4.45 g (90%) of the product as a white foam.

¹H NMR (CDCl₃, 300 MHz) δ 7.15-6.90 (m, 3H), 6.60 (bs, 1H), 4.40 (d, J=5.1 Hz, 2H), 4.40-4.15 (m, 2H), 3.50-3.35 and 2.90-2.75 (m, total 1H), 2.55-2.45 (m, 1H), 2.15-1.55 (m, 7H), 1.50 (s, 9H);

19F NMR (CDCl₃, 282 MHz) δ −75.34 (s, 3F), −117.06 and −118.66 (total, 1F);

LCMS 1.08 min m/z: [M−H]⁺=429.

### Step F

### N-[3-(8-aza-bicyclo[3.2.1]oct-3-yl)-4-fluoro-benzyl]-2,2,2-trifluoro-acetamide hydrochloride

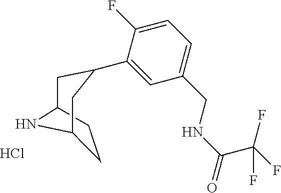

A mixture of 3-{2-fluoro-5-[(2,2,2-trifluoro-acetylamino)-methyl]-phenyl}-8-aza-bicyclo[3.2.1]octane-8-carboxylic acid tert-butyl ester (4.45 g, 10.3 mmol) in 4M HCl in dioxane (50 mL) was stirred at r.t. overnight. The mixture was concentrated to dryness, and the residue was co-evaporated with Et₂O (2×) to yield 4.15 g of the crude product as a white foam.

¹H NMR (CDCl₃, 300 MHz) δ 9.95-9.40 (m, 2H), 7.80-6.70 (m, 3H), 4.60-4.35 (m, 2H), 4.30-4.05 (m, 2H), 3.85-3.35 (m, 2H), 2.85-2.20 (m, 3H), 2.20-1.70 (m, 5H);

¹⁹F NMR (CDCl₃, 282 MHz) δ −75.19 and −75.32 (total, 3F), −114.78 and −120.38 (total, 1F);

LCMS 0.59 min m/z: [M+H]⁺=331.

### Step G

### 2,2,2-Trifluoro-N-(4-fluoro-3-{8-[1-(2-methoxy-ethyl)-7-methyl-1H-indole-3-carbonyl]-8-aza-bicyclo[3.2.1]oct-3-yl}-benzyl)-acetamide

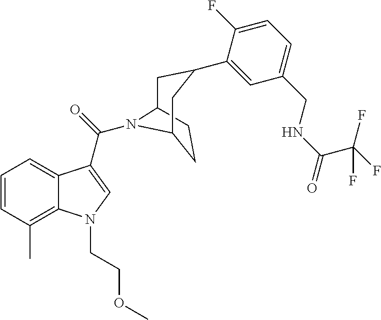

A mixture of N-[3-(8-aza-bicyclo[3.2.1]oct-3-yl)-4-fluoro-benzyl]-2,2,2-trifluoro-acetamide hydrochloride (940 mg, 2.56 mmol), 1-(2-methoxy-ethyl)-7-methyl-1H-indole-3-carboxylic acid (619 mg, 2.64 mmol), TEA (1.2 mL, 8.80 mmol), and EDCl (540 mg, 3.10 mmol) in CH₂Cl₂ (50 mL) was stirred at r.t. overnight. The mixture was partitioned between H₂O and CH₂Cl₂. The two layers were separated, and the organic layer was washed with brine, dried over MgSO₄, filtered, and concentrated in vacuo. The crude material was purified on silica gel with heptane/EtOAc (60/40 to 0/100) to yield two product conformers.

Conformer 1: white solid (480 mg, 40%), higher Rf/lower Rf isomers ratio (80/20).

¹H NMR (CDCl₃, 300 MHz) δ 7.80-7.60 (m, 1H), 7.46 (s, 1H), 7.20-6.95 (m, 5H), 6.81 (bs, 1H), 5.00-4.30 (m, 6H), 3.70 (t, J=5.6 Hz, 2H), 3.65-3.45 (m, 1H), 3.29 (s, 3H), 2.71 (s, 3H), 2.25-1.40 (m, 8H);

¹⁹F NMR (CDCl₃, 282 MHz) δ −75.27 and −75.29 (total, 3F), −117.00 and −118.93 (total, 1F);

LCMS 1.03 min m/z: [M+H]⁺=546.

Conformer 2: white solid (440 mg, 36%), higher Rf/lower Rf isomers ratio (25/75). ¹H NMR (CDCl₃, 300 MHz) δ 7.80-7.60 (m, 1H), 7.46 (s, 1H), 7.20-6.95 (m, 5H), 6.82 (bs, 1H), 5.10-4.35 (m, 6H), 3.70 (t, J=5.4 Hz, 2H), 3.30 (s, 3H), 3.20-3.00 (m, 1H), 2.71 (s, 3H), 2.60-2.35 (m, 1H), 2.25-1.40 (m, 7H);

¹⁹F NMR (CDCl₃, 282 MHz) δ −75.26 and −75.30 (total, 3F), −116.98 and −118.95 (total, 1F);

LCMS 1.02 min m/z: [M+H]⁺=546.

### Step H

### [3-(5-Aminomethyl-2-fluoro-phenyl)-8-aza-bicyclo[3.2.1]oct-8-yl]-[1-(2-methoxy-ethyl)-7-methyl-1H-indol-3-yl]-methanone hydrochloride

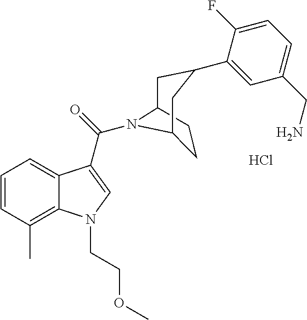

A mixture of 2,2,2-trifluoro-N-(4-fluoro-3-{8-[1-(2-methoxy-ethyl)-7-methyl-1H-indole-3-carbonyl]-8-aza-bicyclo[3.2.1]oct-3-yl}-benzyl)-acetamide conformer 1 (480 mg, 0.88 mmol) and potassium carbonate (1.21 g, 8.8 mmol) in MeOH/H₂O (25 mL/10 mL) was stirred at r.t. overnight. The mixture was concentrated in vacuo, and the residue was partitioned between H₂O and EtOAc. The two layers were separated, and the aqueous layer was re-extracted with EtOAc (2×). The combined organic extracts were washed with brine, dried over MgSO4, filtered, and concentrated in vacuo. The residue was suspended in Et₂O, and 2M HCl in Et₂O was added. The suspension was concentrated to dryness to yield 410 mg (95%) of the product as a white powder.

¹H NMR (CDCl₃, 300 MHz) δ 9.35-8.75 (m, 3H), 8.15-7.40 (m, 3H), 7.25-6.80 (m, 4H), 4.80-3.95 (m, 6H), 3.80-3.30 (m, 3H), 3.25 (s, 3H), 2.56 (s, 3H), 2.20-1.50 (m, 8H);

¹⁹F NMR (CDCl₃, 282 MHz) δ −116.82 and −118.65 (total, 1F);

LCMS 0.74 min m/z: [M+H]⁺=450.

### Example 2

### [3-(5-Aminomethyl-2-fluoro-phenyl)-8-aza-bicyclo[3.2.1]oct-8-yl]-[1-(2-methoxy-ethyl)-7-trifluoromethoxy-1H-indol-3-yl]-methanone hydrochloride

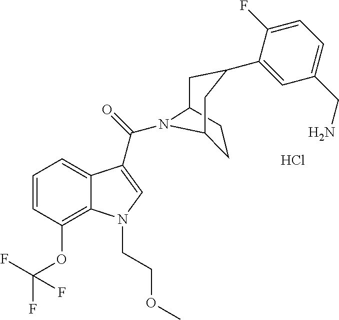

### Step A

### 2,2,2-Trifluoro-N-(4-fluoro-3-{8-[1-(2-methoxy-ethyl)-7-trifluoromethoxy-1H-indole-3-carbonyl]-8-aza-bicyclo[3.2.1]oct-3-yl}-benzyl)-acetamide

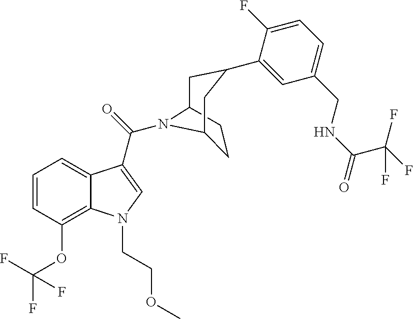

A mixture of N-[3-(8-aza-bicyclo[3.2.1]oct-3-yl)-4-fluoro-benzyl]-2,2,2-trifluoro-acetamide hydrochloride (330 mg, 0.9 mmol), 1-(2-methoxy-ethyl)-7-trifluoromethoxy-1H-indole-3-carboxylic acid (303 mg, 1.0 mmol), TEA (0.28 mL, 2.0 mmol), and EDCl (230 mg, 1.2 mmol) in CH₂Cl₂ (10 mL) was stirred at r.t. overnight. The mixture was partitioned between H₂O and CH₂Cl₂. The two layers were separated, and the organic layer was washed with brine, dried over MgSO₄, filtered, and concentrated in vacuo. The crude material was purified on silica gel with heptane/EtOAc (70/30 to 40/60) to yield two product conformers.

Conformer 1: white solid (210 mg, 38%), higher Rf/lower Rf isomers ratio (94/6).

¹H NMR (CDCl₃, 300 MHz) δ 7.90-7.75 (m, 1H), 7.51 (s, 1H), 7.25-6.90 (m, 5H), 6.81 (bs, 1H), 5.00-4.60 (m, 2H), 4.55-4.35 (m, 4H), 3.71 (t, J=5.2 Hz, 2H), 3.60-3.40 (m, 1H), 3.29 (s, 3H), 2.25-1.65 (m, 8H);

¹⁹F NMR (CDCl₃, 282 MHz) δ −56.38 (s, 3F), −75.30 (s, 3F), −118.79 (s, 1F);

LCMS 1.10 min m/z: [M+H]⁺=616.

Conformer 2: white solid (130 mg, 23%), higher Rf/lower Rf isomers ratio (35/65).

¹H NMR (CDCl₃, 300 MHz) δ 7.90-7.75 (m, 1H), 7.52 (s, 1H), 7.20-6.90 (m, 5H), 6.62 (bs, 1H), 5.00-4.35 (m, 6H), 3.72 (t, J=5.1 Hz, 2H), 3.30 (s, 3H), 3.20-3.00 (m, 1H), 2.65-2.35 (m, 1H), 2.25-1.40 (m, 7H);

¹⁹F NMR (CDCl₃, 282 MHz) δ −56.39 (s, 3F), −75.30 and −75.33 (total, 3F), −116.94 and −118.76 (total, 1F);

LCMS 1.09 min m/z: [M+H]⁺=616.

### Step B

### [3-(5-Aminomethyl-2-fluoro-phenyl)-8-aza-bicyclo[3.2.1]oct-8-yl]-[1-(2-methoxy-ethyl)-7-trifluoromethoxy-1H-indol-3-yl]-methanone hydrochloride

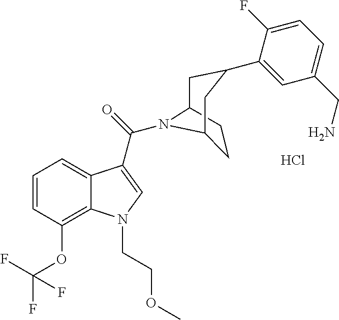

A mixture of 2,2,2-trifluoro-N-(4-fluoro-3-{8-[1-(2-methoxy-ethyl)-7-trifluoromethoxy-1H-indole-3-carbonyl]-8-aza-bicyclo[3.2.1]oct-3-yl}-benzyl)-acetamide conformer 1 (210 mg, 0.34 mmol) and potassium carbonate (0.49 g, 3.4 mmol) in MeOH/H₂O (10 mL/4 mL) was stirred at r.t. overnight. The mixture was concentrated in vacuo, and the residue was partitioned between H₂O and EtOAc. The two layers were separated, and the aqueous was re-extracted with EtOAc (2×). The combined organic extracts were washed with brine, dried over MgSO₄, filtered, and concentrated in vacuo. The residue was suspended in Et₂O and 2M HCl in Et₂O was added. The suspension was concentrated to dryness to yield 185 mg (97%) of the product as a white powder.

¹H NMR (DMSO-d₆, 300 MHz) δ 8.28 (bs, 3H), 8.00-7.85 (m, 2H), 7.60-7.45 (m, 1H), 7.40-7.30 (m, 1H), 7.30-7.10 (m, 4H), 4.69 (m, 2H), 4.50 (t, J=5.3 Hz, 2H), 4.10-3.90 (m, 2H), 3.80-3.40 (m, 3H), 3.21 (s, 3H), 2.20-1.70 (m, 8H);

¹⁹F NMR (CDCl₃, 282 MHz) δ −56.32 and −56.33 (total 3F), −118.61 (s, 1F);

LCMS 0.79 min m/z: [M+H]⁺=520.

### Example 3

### [3-(5-Aminomethyl-2-fluoro-phenyl)-8-aza-bicyclo[3.2.1]oct-8-yl]-[4-fluoro-1-(2-methoxy-ethyl)-7-methyl-1H-indol-3-yl]-methanone hydrochloride

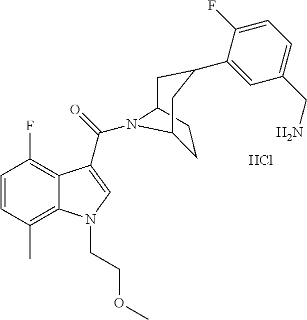

### Step A

### 2,2,2-Trifluoro-N-(4-fluoro-3-{8-[4-fluoro-1-(2-methoxy-ethyl)-7-methyl-1H-indole-3-carbonyl]-8-aza-bicyclo[3.2.1]oct-3-yl}-benzyl)-acetamide

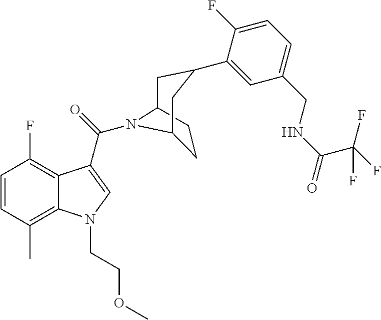

A mixture of N-[3-(8-aza-bicyclo[3.2.1]oct-3-yl)-4-fluoro-benzyl]-2,2,2-trifluoro-acetamide hydrochloride (366 mg, 1.0 mmol), 4-fluoro-1-(2-methoxy-ethyl)-7-methyl-1H-indole-3-carboxylic acid (256 mg, 1.0 mmol), TEA (0.28 mL, 2.0 mmol), and EDCl (250 mg, 1.3 mmol) in CH₂Cl₂ (10 mL) was stirred at r.t. overnight. The mixture was partitioned between H₂O and CH₂Cl₂. The two layers were separated, and the organic layer was washed with brine, dried over MgSO₄, filtered, and concentrated in vacuo. The crude material was purified on silica gel with heptane/EtOAc (50/50 to 0/100) to yield two product conformers. The yield of the reaction was 460 mg (81%).

Higher Rf/lower Rf isomers ratio (94/6).

¹H NMR (CDCl₃, 300 MHz) δ 7.40-7.30 (m, 1H), 7.25-6.80 (m, 6H), 5.00-4.75 and 4.30-4.05 (m, 2H), 4.60-4.30 (m, 4H), 3.75-3.60 (m, 2H), 3.60-3.40 and 3.15-2.95 (m, 1H), 3.29 and 3.28 (s, 3H), 2.60 (s, 3H), 2.40-1.40 (m, 8H);

¹⁹F NMR (CDCl₃, 282 MHz) δ −75.26 and −75.31 (total, 3F), −116.80 and −119.35 (total, 1F), −22.97 and −123.33 (total, 1F);

LCMS 1.03 and 1.04 min m/z: [M+H]⁺=564.

### Step B

### [3-(5-Aminomethyl-2-fluoro-phenyl)-8-aza-bicyclo[3.2.1]oct-8-yl]-[4-fluoro-1-(2-methoxy-ethyl)-7-methyl-1H-indol-3-yl]-methanone hydrochloride

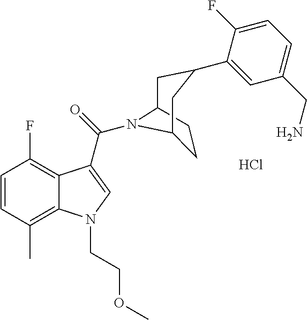

A mixture of 2,2,2-trifluoro-N-(4-fluoro-3-{8-[4-fluoro-1-(2-methoxy-ethyl)-7-methyl-1H-indole-3-carbonyl]-8-aza-bicyclo[3.2.1]oct-3-yl}-benzyl)-acetamide (450 mg, 0.84 mmol) and potassium carbonate (1.16 g, 8.4 mmol) in MeOH/H₂O (20 mL/8 mL) was stirred at r.t. overnight. The mixture was concentrated in vacuo, and the residue was partitioned between H₂O and EtOAc. The two layers were separated, and the aqueous was re-extracted with EtOAc (2×). The combined organic extracts were washed with brine, dried over MgSO₄, filtered, and concentrated in vacuo. The residue was suspended in Et₂O, and 2M HCl in Et₂O was added. The suspension was concentrated to dryness to yield 340 mg (80%) of the product as a white powder.

¹H NMR (CDCl₃, 300 MHz) δ 9.20-8.50 (m, 3H), 8.20-6.55 (m, 6H), 4.90-3.85 (m, 6H), 3.80-3.35 (m, 3H), 3.30-3.00 (m, 3H), 2.80-2.40 (m, 3H), 2.30-1.40 (m, 8H);

¹⁹F NMR (CDCl₃, 282 MHz) δ −116.36 and −119.63 (total 1F), −123.14 and 124.07 (total, 1F);

LCMS 0.72, 0.74 min m/z: [M+H]⁺=468.

### Example 4

### [3-(5-Aminomethyl-2-fluoro-phenyl)-8-aza-bicyclo[3.2.1]oct-8-yl]-(4-bromo-3-methyl-5-propoxy-thiophen-2-yl)-methanone hydrochloride

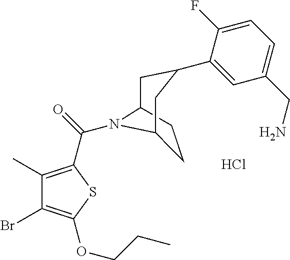

### Step A

### N-{3-[8-(4-Bromo-3-methyl-5-propoxy-thiophene-2-carbonyl)-8-aza bicyclo[3.2.1]oct-3-yl]-4-fluoro-benzyl}-2,2,2-trifluoro-acetamide

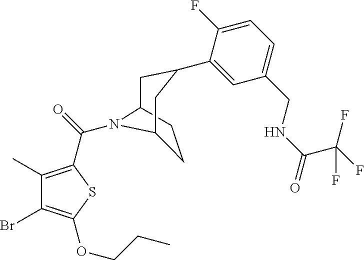

A mixture of N-[3-(8-aza-bicyclo[3.2.1]oct-3-yl)-4-fluoro-benzyl]-2,2,2-trifluoro-acetamide hydrochloride (400 mg, 1.09 mmol), 4-bromo-3-methyl-5-propoxy-thiophene-2-carboxylic acid (365 mg, 1.3 mmol), TEA (0.30 mL, 2.8 mmol), and EDCl (272 mg, 1.4 mmol) in CH₂Cl₂ (20 mL) was stirred at r.t. overnight. The mixture was portioned between H₂O and CH₂Cl₂. The two layers were separated and the organic layer was washed with brine, dried over MgSO₄, filtered, and concentrated in vacuo. The crude material was purified on silica gel with heptane/EtOAc (80/20 to 50/50) to yield two product conformers.

Conformer 1: white solid (203 mg, 31%), higher Rf/lower Rf isomers ratio (90/10).

¹H NMR (CDCl₃, 300 MHz) δ 7.20-6.90 (m, 3H), 6.53 (bs, 1H), 4.65-4.40 (m, 4H), 4.08 (t, J=6.6 Hz, 2H), 3.60-3.35 (m, 1H), 2.27 (s, 3H), 2.20-1.65 (m, 10H), 1.05 (t, J=7.4 Hz, 3H);

¹⁹F NMR (CDCl₃, 282 MHz) δ −76.21 and −76.25 (total, 3F), −119.47 and −119.45 (total, 1F);

LCMS 1.14 min m/z: [M+H]⁺=591.

Conformer 2: white solid (120 mg, 20%), higher Rf/lower Rf isomers ratio (30/70).

¹H NMR (CDCl₃, 300 MHz) δ 7.20-6.90 (m, 3H), 6.51 (bs, 1H), 4.65-4.40 (m, 4H), 4.09 (t, J=6.6 Hz, 2H), 3.10-2.90 (m, 1H), 2.60-2.45 (m, 2H), 2.31 (s, 3H), 2.20-1.70 (m, 8H), 1.06 (t, J=7.3 Hz, 3H);

¹⁹F NMR (CDCl₃, 282 MHz) δ −76.22 and −76.25 (total, 3F), −117.64 (s, 1F);

LCMS 1.14 min m/z: [M+H]⁺=591.

### Step B

### [3-(5-Aminomethyl-2-fluoro-phenyl)-8-aza-bicyclo[3.2.1]oct-8-yl]-(4-bromo-3-methyl-5-propoxy-thiophen-2-yl)-methanone hydrochloride

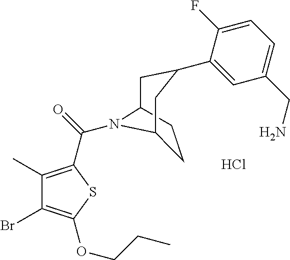

A mixture of N-{3-[8-(4-bromo-3-methyl-5-propoxy-thiophene-2-carbonyl)-8-aza bicyclo[3.2.1]oct-3-yl]-4-fluoro-benzyl}-2,2,2-trifluoro-acetamide conformer 1 (203 mg, 0.34 mmol) and potassium carbonate (0.38 g, 2.7 mmol) in MeOH/H₂O (20 mL/4 mL) was stirred at r.t. overnight. The mixture was concentrated in vacuo, and the residue was partitioned between H₂O and EtOAc. The two layers were separated, and the aqueous was re-extracted with EtOAc (2×). The combined organic extracts were washed with brine, dried over MgSO₄, filtered, and concentrated in vacuo. The residue was suspended in Et₂O, and 2M HCl in Et₂O was added. The suspension was concentrated to dryness to yield 150 mg (88%) of the product as a white powder.

¹H NMR (DMSO-d₆, 300 MHz) δ 8.22 (bs, 3H), 7.55-7.15 (m, 3H), 4.42 (s, 2H), 4.12 (t, J=6.4 Hz, 2H), 4.10-3.90 (m, 2H), 3.55-3.35 (m, 1H), 2.21 (s, 3H), 2.10-1.60 (m, 10H), 0.98 (t, J=7.3 Hz, 3H);

¹⁹F NMR (CDCl₃, 282 MHz) δ −118.76 and −119.10 (total, 1F);

LCMS 0.86 min m/z: [M+H]⁺=495.

### Biological Activity

The properties of the compound of the present invention are demonstrated by its beta-Tryptase Inhibitory potency (IC₅₀ and Kᵢ values).

The compounds of this invention display affinity constants (Ki) in the range of 1 μM to 60 nM.

### In Vitro Test Procedure

As all the actions of tryptase, as described in the background section, are dependent on its catalytic activity, then compounds that inhibit its catalytic activity will potentially inhibit the actions of tryptase. Inhibition of this catalytic activity may be measured by the in vitro enzyme assay and the cellular assay.

Tryptase inhibition activity is confirmed using either isolated human lung tryptase or recombinant human beta tryptase expressed in yeast cells. Essentially equivalent results are obtained using isolated native enzyme or the expressed enzyme. The assay procedure employs a 96 well microplate (Costar 3590) using L-pyroglutamyl-L-prolyl-L-arginine-para-nitroanilide (S2366: Quadratech) as substrate (essentially as described by McEuen et. al. Biochem Pharm, 1996, 52, pages 331-340). Assays are performed at room temperature using 0.5 mM substrate (2×Kₘ) and the microplate is read on a microplate reader (Beckman Biomek Plate reader) at 405 nm wavelength.

### Materials and Methods for Tryptase Primary Screen (Chromogenic Assay)

### Assay Buffer

50 mM Tris (pH 8.2), 100 μM NaCl, 0.05% Tween 20, 50 μg/mL heparin.

### Substrate

S2366 (Stock solutions of 2.5 μM).

### Enzyme

Purified recombinant beta Tryptase Stocks of 310 μg/mL.

### Protocol (Single Point Determination)

Add 60 μL of diluted substrate (final concentration of 500 μM in assay buffer) to each well
Add compound in duplicates, final concentration of 20 μM, volume 20 μL
Add enzyme at a final concentration of 50 ng/mL in a volume of 20 μL
Total volume for each well is 100 μL
Agitate briefly to mix and incubate at room temp in the dark for 30 minutes
Read absorbencies at 405 nM
   Each plate has the following controls:
Totals: 60 μL of substrate, 20 μL of buffer (with 0.2% final concentration of DMSO),
   20 μL of enzyme
Non-specific: 60 μL of substrate, 40 μL of buffer (with 0.2% DMSO)
Totals: 60 μL of substrate, 20 μL of buffer (No DMSO), 20 μL of enzyme
Non-specific: 60 μL of substrate, 40 μL of buffer (No DMSO)

### Protocol IC₅₀ and Kᵢ Determination)

The protocol is essentially the same as above except that the compound is added in duplicates at the following final concentrations: 0.01, 0.03, 0.1, 0.3, 1, 3, 10 μM (All dilutions carried out manually). For every assay, whether single point or IC₅₀ determination, a standard compound is used to derive IC₅₀ for comparison. From the IC₅₀ value, the Kᵢ can be calculated using the following formula: Kᵢ=IC₅₀/(1+[Substrate]/Kₘ).

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A compound of formula (I): 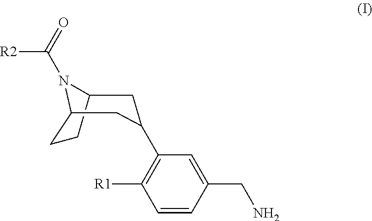
wherein
R1 is F, Cl, Br, —OCH₂CO₂CH₃, —CH₂OH, or an alkyl, haloalkyl, alkoxy or haloalkoxy group; and
R2 is aryl or heteroaryl that is optionally substituted; or
a pharmaceutically acceptable salt thereof or an enantiomer or a diastereomer thereof.

2. The compound according to claim 1, wherein
R1 is selected from the group consisting of F, Cl, Br, OCH₂CO₂CH₃ and CH₂OH.

3. The compound according to claim 1, wherein 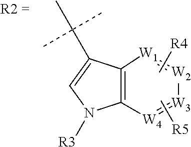
wherein
R3 is alkyl, or alkyl optionally substituted by one or more groups selected from hydroxy, alkoxy, haloalkoxy, cycloalkyl, heterocycle, aryl, optionally substituted aryl, heteroaryl or optionally substituted heteroaryl;
R4 and R5 are each independently H, halo, alkoxy, haloalkoxy, alkyl, amido, ureyl, carboxyl, sulfonyl amido, sulfonyl urea, alkyl optionally substituted by one or more groups selected from hydroxy, alkoxy, haloalkoxy, cycloalkyl, heterocycloalkyl, aryl and heteroaryl; and
W₁, W₂, W₃ and W₄ are each N, CH, CR4 or CR5.

4. The compound according to claim 1, wherein R2 is indolyl or thiophenyl that is optionally substituted.

5. The compound of claim 1 selected from the group consisting of:
[3-(5-Aminomethyl-2-fluoro-phenyl)-8-aza-bicyclo[3.2.1]oct-8-yl]-[1-(2-methoxy-ethyl)-7-methyl-1H-indol-3-yl]-methanone hydrochloride;
[3-(5-Aminomethyl-2-fluoro-phenyl)-8-aza-bicyclo[3.2.1]oct-8-yl]-[1-(2-methoxy-ethyl)-7-trifluoromethoxy-1H-indol-3-yl]-methanone hydrochloride;
[3-(5-Aminomethyl-2-fluoro-phenyl)-8-aza-bicyclo[3.2.1]oct-8-yl]-[4-fluoro-1-(2-methoxy-ethyl)-7-methyl-1H-indol-3-yl]-methanone hydrochloride; and
[3-(5-Aminomethyl-2-fluoro-phenyl)-8-aza-bicyclo[3.2.1]oct-8-yl]-(4-bromo-3-methyl-5-propoxy-thiophen-2-yl)-methanone hydrochloride; or
a salt thereof or an enantiomer or a diastereomer thereof.

6. A pharmaceutical composition comprising a compound according to claim 1, in combination with at least one pharmaceutically acceptable excipient, diluent or carrier.

7. A method of treating an inflammatory disease in a patient, comprising administering to said patient a therapeutically effective amount of a compound according to claim 1.

8. The method according to claim 6 wherein the inflammatory disease is COPD.
